(19) United States
(12) Patent Application Publication
(10) Pub. No.: US 2015/0073930 A1
(43) Pub. Date: Mar. 12, 2015

(54) ADHESIVE BANDAGE

(71) Applicant: Odette Perreault, Long Beach, CA (US)

(72) Inventor: Odette Perreault, Long Beach, CA (US)

(21) Appl. No.: 14/025,502

(22) Filed: Sep. 12, 2013

(51) Int. Cl. A61F 13/00; G06Q 30/06; A61F 13/02

(52) U.S. Cl. CPC A61F 13/00059; A61F 13/0243; A61F 13/0283; G06Q 30/0621; A61F 2013/00153
USPC 705/26.5; 602/57; 602/42

(57) ABSTRACT

An adhesive bandage is provided. The adhesive bandage compromises a strip having an upper surface and a lower surface; a wound-contacting pad disposed on the lower surface; at least one protective release tab removably disposed on the lower surface; wherein at least a portion of the upper surface is manufactured to match a particular user's skin tone.

### FIELD

This invention relates to adhesive bandages, and more specifically, to adhesive bandages precisely matched to a user's skin tone.

### BACKGROUND

Adhesive bandages, such as “band aids” are commonly applied to a wound. These adhesive bandages are generally a single color, i.e., a single brownish color. Generally, a plurality of such single-color brownish adhesive bandages is packaged together. The user does not get a choice of exactly which color he or she would like the adhesive bandage to be. As such, users that are fair-skinned may have to use an adhesive bandage that is much darker than their skin, and users that are dark-skinned may have to use an adhesive bandage that is much lighter than their skin. This may be troublesome for those in certain professions, as the adhesive bandages may look aesthetically unappealing on the user's skin. For example, a light-colored adhesive bandage on a darker-skinned model may look aesthetically unappealing and/or may prevent the model from working when he or she has one or more adhesive bandages disposed on his or her skin. Accordingly, there is a need for adhesive bandages that are matched to a user's skin tone/color.

### BRIEF SUMMARY OF THE INVENTION

In one embodiment, an adhesive bandage is provided. The adhesive bandage includes a strip having an upper surface and a lower surface and at least one protective release tab removably disposed on the lower surface. At least a portion of the upper surface matches a particular user's skin tone. Preferably, the adhesive bandage is made-to-order. Preferably, the Pantone SkinTone™ Guide is used to identify the particular user's skin tone. In some embodiments, the made-to-order adhesive bandage is a silicone bandage. In other embodiments, the adhesive bandage is not made-to-order, i.e., certain common skin tone colors may be purchased immediately on-line or on a store shelf.

In one embodiment, a method of providing an adhesive bandage matched to a particular user's skin tone is provided. The method includes the step of providing a sample of colors to a retailer and matching the particular user's skin tone to a color from the sample at the retailer. The method may include the step of ordering the adhesive bandage with a selected color. In one aspect, the method may include the step of providing the adhesive bandage with a selected color.

In one embodiment, a method of providing an adhesive bandage matched to a particular user's skin tone is provided. The method includes the step of identifying skin tone colors in a particular geographic region; and providing an adhesive bandage in skin tone colors to a retailer based upon the particular geographic region, wherein the adhesive bandage is provided in a package having an opening. In one aspect, the method includes the step of holding the user's skin to the opening and thereby determining whether the user's skin tone matches the adhesive bandage contained therein.

In one embodiment, a method of providing an adhesive bandage precisely matched to a particular user's skin tone is provided. The method includes the steps of receiving information regarding the particular user's skin tone and providing the adhesive bandage in a selected color, wherein the selected color is determined by using one or more color swatches.

In one embodiment, a kit is provided. The kit includes a sample of colors and instructions for ordering an adhesive bandage based upon a selected color.

In one embodiment, a made-to-order wound tape or gauze is provided. The made-to-order wound tape or gauze includes an upper surface and a lower surface. At least a portion of the upper surface matches a particular user's skin tone.

### BRIEF DESCRIPTION OF THE DRAWINGS

FIG. 1 is a top plan view of a preferred embodiment of the adhesive bandage of the present invention;

FIG. 2 is a bottom plan view of a preferred embodiment of the adhesive bandage of the present invention;

FIG. 3 is a bottom plan view of a preferred embodiment of the adhesive bandage of the present invention;

FIG. 4 is a view of a plastic sheaths according to one embodiment of the present invention; and

FIG. 5 is a bottom plan view of another preferred embodiment of the adhesive bandage of the present invention.

### DETAILED DESCRIPTION OF THE INVENTION

The present invention is directed to an adhesive bandage that is manufactured, made, and/or provided to preferably precisely match the user's skin tone using a skin tone guide, using, for example, but not limited to, the Pantone SkinTone™ Guide.

### DEFINITIONS

“Matches a particular user's skin tone,” “matches a user's skin tone,” “made/manufactured to match a [particular] user's skin tone,” and/or “precisely matches” (or any grammatical version thereof) means that the product is made to match that particular user's skin/skin tone after determining/identifying that particular user's skin tone using the methods described herein. Skin tone can be determined by using color swatches (i.e., color samples), colorimeters, plastic sheaths and/or other skin tone color identification methods known in the art. The product may be made-to-order and/or the product may be available at the retailer and/or distributor. If the product is available at the retailer, the user can match his or her skin tone to the product itself at the retailer.

“Skin tone” may refer to a person's skin tone, skin hue, and/or skin hue and skin tone. “Skin tone” is interchangeable with “skin color.”

“Adhesive bandage” may refer to a single bandage, or may refer to more than one bandage. The term may refer to a single type of bandages or many different types of bandages. It may also refer to a “silicone bandage.” In some embodiments, “adhesive bandage” may refer to wound tape or gauze.

### Adhesive Bandage

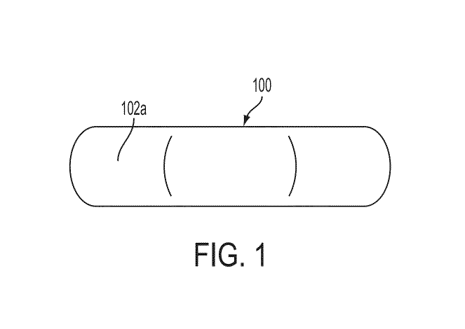

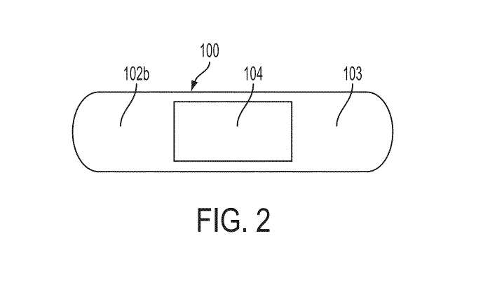

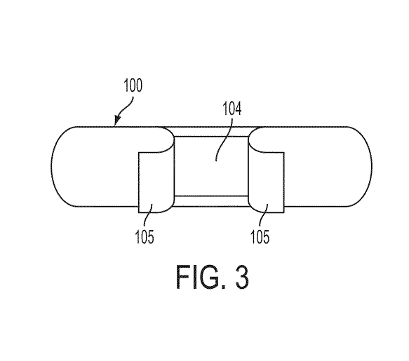

In one embodiment, and as shown in FIGS. 1-5, an adhesive bandage 100 (or “band-aid”) is provided. Adhesive bandage 100 preferably includes a strip 102 having an upper surface 102a and a lower surface 102b, an adhesive 103, an absorbent, wound-contacting pad 104 (“wound-contacting pad” or “absorbent pad”), and a pair of protective release tabs 105 (identified singularly and plurally as “105”). At least a portion of lower surface 102b contacts a user's skin. Adhesive 103 is at least partially disposed on lower surface 102b. Wound-contacting pad 104 is disposed on lower surface 102b. Preferably, protective release tabs 105 are removably disposed on lower surface 102b. In other embodiments, strip 102 may compromise a single protective release tab 105, or strip 102 may compromise more than a pair of protective release tabs 105. In yet other embodiments, adhesive 103; protective release tabs 105; and/or wound-contacting pads 104 are omitted. For example, adhesive bandage may be a silicone bandage comprised/consisting essentially of/consisting of silicone. In this embodiment, wound-contacting pad 104 is preferably omitted. It is to be understood that the present invention can be used with any adhesive bandage now known or later contemplated.

Absorbent pad 104 is typically a nonwoven material made from rayon fibers or polyester fibers or a blend of such fibers. It will be apparent to those skilled in the art that other fibers, e.g., cotton fibers or polyolefin fibers may be used in constructing absorbent pad 104. Protective release tabs 105 may be made from paper having a silicone release material coated thereon and/or may be made of a low surface energy plastic film such as polyethylene or polystyrene which, if desired, may have a silicone release material or the like applied thereto. The release coated surface of protective release tabs 105 will contact adhesive 103 and the release coating on protective release tabs 105 will be such that adhesive 103 will remain on lower surface 102b rather than on protective release tabs 105 when those tabs are removed to expose adhesive 103 prior to application of adhesive bandage 100 to the skin. Adhesive 103 is made from any skin-compatible material known in the art, i.e., adhesive free of latex, and/or substantially free of latex.

Adhesive bandage 100 and/or strip 102 may be provided in any shape and/or configuration as is known in the art. The shape of adhesive bandage 100 may dictate the number of protective release tabs 105 it compromises, and/or location of wound-contacting pad 104 on lower surface 102b. For example, if strip 102 is rectangular, wound-contacting pad 104 is preferably in the middle, or substantially middle, of strip 102. If strip 102 is circular, for example, adhesive bandage 100 may have a single protective release tab 105. Further, adhesive bandage 100 may be comprised of any material, such as silicone, fabric, plastic, and the like.

The adhesive bandages 100 of the present invention may be individually packaged between two sheets of paper which are sealed cohesively about the edges.

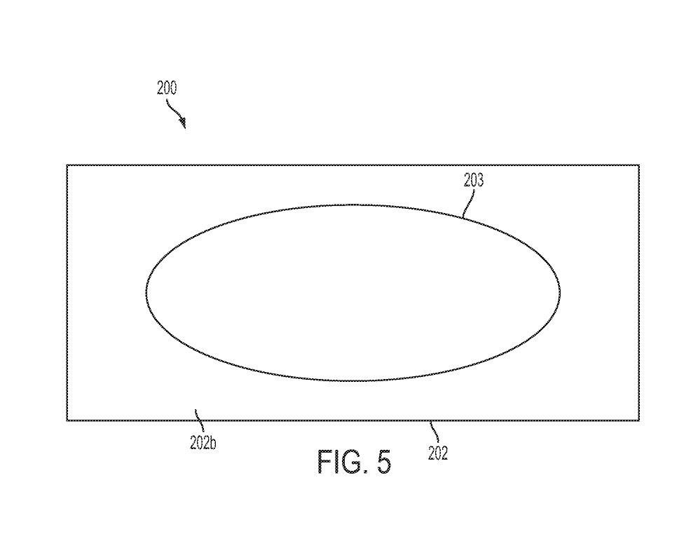

In one embodiment, and referring to FIG. 5, adhesive bandage is a silicone bandage 200. Silicone bandages are known in the art and may be used for scar and/or burn treatment. In this embodiment, adhesive bandage preferably includes strip 202 having an upper surface 202a (not shown), a lower surface 202b, and an adhesive 203. Adhesive 203 may be disposed on lower surface 202b and may occupy entire lower surface 202b or a portion of lower surface 202b. Preferably, a wound-contacting pad is omitted. In this regard, silicone bandage may be attached to user's skin using medical tape, self-adhesive tape, and/or via adhesive 203 disposed on lower surface 202b. Preferably, silicone bandage is comprised of; consists essentially of; or consists of silicone. In some embodiments, upper surface 202a (not shown) is comprised of silicone. In some embodiments, a top layer 206 (not shown) is disposed, at least partially, on upper surface 202a (not shown). In these embodiments, upper surface 202a and/or top layer 206 may be made to precisely match a user's skin using the methods described herein. In some embodiments, silicone bandage 200 may be a sheath of silicone that may be cut to the appropriate size and/or shape and/or secured to the user's skin or other surface with tape, self-adherent tape and/or medical wrap. In some embodiments, lower surface 202b may be comprised of, consist of, and/or consist essentially of, an adhesive. In some embodiments, adhesive 203 may be comprised of, consist of, and/or consist essentially of, silicone. In this regard, the silicone may have an adhesive quality (i.e., a clinging quality), but may not necessarily stick to the skin or other surface. In some embodiments, adhesive 203 is omitted. In some embodiments, silicone bandage 200 may be available from, but not limited to, NuSil Technology LLC (Carpenteria, Calif.) or Applied Silicone Corporation (Santa Paula, Calif.). It is contemplated that any other silicone bandage now known or later contemplated may be precisely matched to a user's skin using the methods described herein. In this regard, it is contemplated that a top surface (i.e., the surface that faces away from the skin surface) of the silicone bandage may be precisely matched to the user's skin.

### Wound Tape/Gauze/Cover-Up

In one embodiment, the invention is directed to a wound tape and/or gauze that is manufactured, made, and/or provided to match the user's skin tone using a skin tone guide, using, for example, but not limited to, the Pantone SkinTone™ Guide. Wound tape holds dressings or bandages around the wound site. Gauze is a surgical dressing that may be secured by medical tape and/or self-adherent wrap. The wound tape or gauze may have an upper surface and a lower surface. Preferably, the upper surface faces away from the user's skin, and the lower surface faces the user's skin. Preferably, at least a portion of the upper surface matches a particular user's skin.

In one embodiment, the invention is directed to a cover-up manufactured, made, and/or provided to match the user's skin tone using a skin tone guide, using, for example, but not limited to, the Pantone SkinTone™ Guide. As used herein, “cover-up” may refer to a material that is used to cover a blemish, or other undesirable feature, on a person's skin. The cover-up may be a strip having an upper surface, a lower surface, an adhesive, and one or more protective release tabs.

### Skin Tone Matching

In one embodiment, adhesive bandage 100 (or wound tape and/or gauze) is manufactured, made, and/or provided to precisely match a particular user's skin tone. In this regard, the Pantone SkinTone™ Guide (provided by Pantone LLC, a wholly-owned subsidiary of X-Rite, Incorporated) may be used to identify the appropriate (1) hue, or undertone, of the skin and (2) tone, or lightness or darkness, of the skin. The Pantone SkinTone™ Guide is a collection of 110 skin tone shades matched to a color standard. It was created by scientifically measuring thousands of actual skin tones across the full spectrum of skin types. Each Pantone SkinTone number is comprised of a four-digit alphanumeric number. The first two positions represent the hue, or undertone, of the skin. The second two positions represent the tone, or lightness and darkness of the skin. In other embodiments, more or less than 110 skin tone shades are provided.

The user's skin hue and/or tone may be identified a number of different ways. For example, the user may be provided with a swatches (or sampling) of the 110 skin tone shades (or any other number of shades), and he/or she may be able use the swatch to identify his or her skin tone by placing the swatch up against his or her skin. These swatches may be available at the retailer and/or provided to the retailer. The user may then be able to order the type(s) of adhesive bandages that he or she would like (i.e., big, small, rectangular, etc.), as well as the exact color at the retailer or through website. In other embodiments, the adhesive bandages may be matched to the color of the user's skin using guides and/or swatches other than the Pantone SkinTone guide.

In one embodiment, a colorimeter (provided, by for example, but not limited to, Konica Minolta or Pantone) may be provided to identify the user's appropriate skin hue/tone. The colorimeter may be provided by the retailer and/or provided to the retailer.

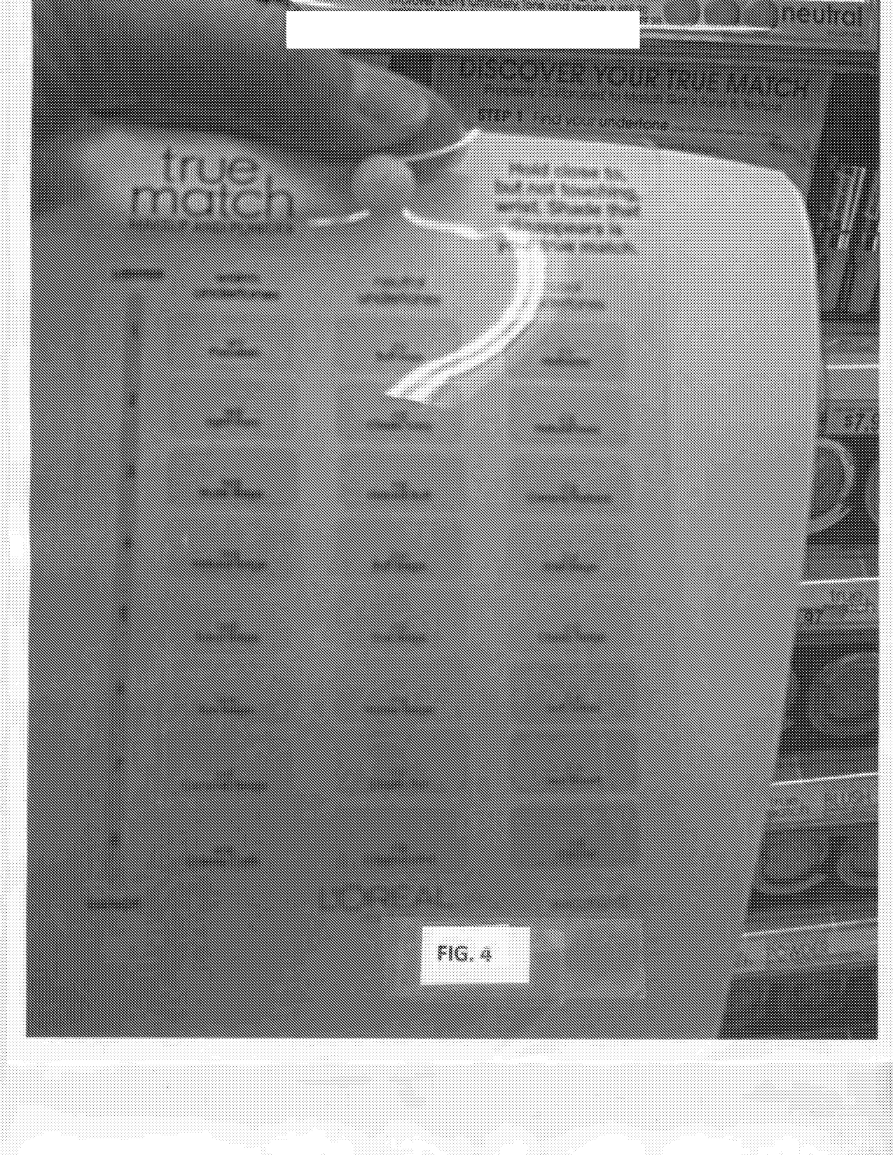

In one embodiment, and as shown in FIG. 4, a set of plastic sheaths (“shade guide”) may be provided to identify the user's hue/tone. These may be located at retailer, provided to the retailer, and/or provided by the retailer. These are held to the user's skin to determine the user's skin tone. These plastic sheaths may be comprised of plastic or other clear material that displays colored sections. The colored sections preferably appear separately from each other on a clear background. A user may thereby position the plastic film over his or her arm, for example, and select among the skin tone shades by identifying the one that appears most consistent with his or her own skin tone that can be seen through the clear background. Each shade may be identified by the corresponding Pantone alpha-numeric code (or other identifying number or feature).

In other embodiments, the user's skin tone may be determined by providing a questionnaire to the user. In yet other embodiments, in any other means to determine skin tone now known or later contemplated may be used without departing from the scope of the present invention.

In some embodiments, the user is provided with a kit containing swatches, as well as sample adhesive bandage(s); wound tape; and/or gauze. The user can select a bandage type as well as color(s) from the kit. The kit may contain instructions for use.

In one embodiment, adhesive bandages (or wound tape and gauzes) may be manufactured with certain selected color(s), packaged, and sold or otherwise distributed at retail stores or any other entity, such as a hospital or a burn center. These color(s) may be selected based upon the geographical location of the retail store wherein the adhesive bandages are to be sold. For example, darker-skinned people may be residing in certain geographical locations. In these locations, the adhesive bandages may be manufactured in darker tones/hues, for example, packaged, and sold at retail stores, or otherwise distributed to other entities. The packaging may have an opening for viewing the adhesive bandages therein in order for the consumer to match the color of the bandage to the color of his or her skin. The opening may be covered by a plastic film or the like. The packaging may also include a removable insert (i.e., removable by tearing along perforation or the like). This insert may include the name of the color of the bandage contained in the packaging, a numerical code identifying the selected color, and/or other identifying feature. The user may easily be able to reorder additional bandages by simply placing the insert in the mail. In other embodiments, the opening is omitted. In this regard, a swatch or other sample of the color of the bandage contained therein may be disposed on an outer surface of the box.

The present invention may be useful for a model, actor/actress, live performer, professional, or the like. For example, a model may have a wound on her arm. If she simply uses a standard bandage, it will likely “show up” against her skin, and it will be aesthetically unappealing. With an adhesive bandage of the present invention, however, a color can be selected that is the same as, or similar to, the color of her skin. As such, it will not “show up” against her skin. Additionally, the present invention may be used, for example, by a child or a professional that does not want to wear unsightly bandages. For example, the professional may play sports or run in his or her free time. It is quite common to have a cut or a bruise playing sports or running The professional may want to wear skirts to work. In this instance, the adhesive bandage of the present invention can be used to cover the wound. It will be less noticeable than a standard bandage, and the professional can wear a skirt to work without drawing too much attention to her wounds that may appear below a garment hem line.

Adhesive Bandage with Skin Tone Matching

In one embodiment, upper surface 102a of adhesive bandage 100 is matched to a user's selected color. As such, the surface that is exposed to the outside (and not in touch with the wound and/or skin) is provided in the user's selected color. It is to be understood, however, that lower surface 102b (portion of adhesive bandage 100 facing the skin); adhesive 103; absorbent, wound-contacting pad 104; and/or protective release tabs 105 may or may not be matched to the user's color.

### EXAMPLE

A user would like to obtain adhesive bandages matching her exact skin tone. She can identify her skin tone(s) by (1) obtaining swatches of skin tone shades and placing the swatch(s) on her skin to identify a match and/or using a colorimeter to identify a color match; (2) ordering and/or purchasing one or more adhesive bandage(s) that are of the identified skin tone. The step of ordering may include logging onto a website to order select the desired adhesive bandage(s) in the identified skin tone(s) and/or calling a number to place the order. The user may have more than one skin tone match. For example, the user may have a first skin tone color on the arm, and a skin tone color on the leg. Any number/type of adhesive bandages may be ordered.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure.

What is claimed is:

1. A made-to-order adhesive bandage comprising:
(a) a strip having an upper surface and a lower surface; and
(b) at least one protective release tab removably disposed on the lower surface; wherein at least a portion of the upper surface matches a particular user's skin tone.

2. The made-to-order adhesive bandage of claim 1, wherein the upper surface is made to match the particular user's skin tone.

3. The made-to-order adhesive bandage of claim 2, wherein one or more color swatches is used to determine the particular user's skin tone.

4. The made-to-order adhesive bandage of claim 3, wherein Pantone SkinTone™ Guide is used to determine the particular user's skin tone.

5. The made-to-order adhesive bandage of claim 4, wherein a colorimeter is used to determine the particular user's skin tone.

6. The made-to-order adhesive bandage of claim 5, further comprising a wound-contacting pad disposed on the lower surface.

7. The made-to-order adhesive bandage of claim 5, wherein the made-to-order adhesive bandage is a silicone bandage.

8. A method of providing an adhesive bandage precisely matched to a particular user's skin tone, compromising:
(a) receiving information regarding the particular user's skin tone; and
(b) providing the adhesive bandage in a selected color, wherein the selected color is determined by using one or more color swatches.

9. The method of claim 8, wherein the selected color is determined by use of Pantone SkinTone™ Guide.

10. The method of claim 8, wherein the selected color is determined by use of a colorimeter.

11. A kit compromising:
(a) a sample of colors; and
(b) instructions for ordering an adhesive bandage based upon a selected color.

12. The kit of claim 11, further compromising Pantone SkinTone™ Guide.

13. The kit of claim 11, further compromising a colorimeter.

14. A made-to-order wound tape or gauze, comprising:
(a) an upper surface; and
(b) a lower surface;
wherein at least a portion of the upper surface matches a particular user's skin tone.

15. The made-to-order wound tape or gauze of claim 14, wherein the upper surface is made to match the particular user's skin tone.

16. The made-to-order wound tape or gauze of claim 12, wherein one or more color swatches is used to determine a particular user's skin tone.

17. The made-to-order wound tape or gauze of claim 12, wherein Pantone SkinTone™ Guide is used to determine the particular user's skin tone.

18. The made-to-order wound tape or gauze of claim 12, wherein a colorimeter is used to determine the particular user's skin tone.
